(19) United States
(12) Patent Application Publication
(10) Pub. No.: US 2002/0023284 A1
(43) Pub. Date: Feb. 28, 2002

(54) High-visibility technical arm-band with pocket

(75) Inventor: Tito Romano, Lago (IT)

Correspondence Address: HARRISON & EGBERT, 7th Floor, 412 Main St., Houston, TX, 77002, US

(73) Assignee: CERBUL SPA

(21) Appl. No.: 09/910,349

(22) Filed: Jul. 23, 2001

(30) Foreign Application Priority Data
Aug. 25, 2000 (IT) TV2000U000040

(51) Int. Cl. A41D 13/08; A61F 9/00

(52) U.S. Cl. 2/16

(57) ABSTRACT

A high-visibility technical arm band with at least one pocket, made up of basically a rectangular strap with reciprocal holding means at each end to form a ring shape, and in which the said strap has a surface made out of high-visibility material on the outer side, and to which a pocket, with an opening which may be opened and closed if required, is joined; the said strap, in correspondence with the rear side which adheres to the underlying surface, has a combination of materials which, distributed in a longitudinal manner, are used to improve the friction with the said surface.

### BACKGROUND OF THE INVENTION

1. Field of the Invention

The object of this invention is a high-visibility arm-band with at least one pocket.

The invention has particular, but not necessarily exclusive, application in the sector involved in accessories for specialised clothing.

2. Description of the Related Art

Articles of clothing used for technical applications are widely known. They are used both in sporting and industrial environments, and each one is optimised according to its specific function.

A large part of these articles of clothing, in both types of application, have a double purpose. On the one hand, they lead to greater comfort, while on the other hand they must meet a series of requirements to facilitate the work of the person who wears the said article of specialised clothing. An important aspect of these requirements is the use of pockets, which are often of an unsuitable dimension, or are distributed in various points of the clothing, for example in the jacket and in the pair of trousers.

A common position of the pockets, in a jacket for example, is along the sleeve close to the shoulder. They are usually made of a size in order to contain small objects, such as a pen, or articles that are more or less the size of a cigarette packet or a pair of glasses.

The purpose of having small pockets for objects of a similar dimension is in order to locate and extract the small object quickly and easily, without having to search and fumble about while looking for the object in the typically larger pockets commonly found in the article of clothing. Furthermore, the conformation and structure of the small pockets thus obtained, for example along the sleeve of the upper part of the limb, means that the items may be distributed in an intelligent fashion in order to easily remember where they have been placed.

The said small pockets may also be either open, or closed with a small strip of material that folds over the front part. The strip is then held in position by buttons, press-studs, Velcro strip, a zip fastener or other such means.

The problems associated with this type of application are due to the fact, when carrying out certain types of work or during sporting activities, it is not always possible to use the specific article of specialised clothing. In a work environment, this is particularly frequent during the summer months when working in hot conditions, since workers often choose to operate without certain articles of clothing, and often resort to the use of only a pair of short trousers. As an alternative, they may choose to wear ordinary articles of clothing that are not suitable for the work being undertaken. The reduction of the surface area of available clothing thus reduced is one of the most common causes, and obliges the person to place the small items in ordinary pockets, which leads to an inefficient use of the items of specialised clothing. The same point may also be made when ordinary articles of clothing are used, which do not usually have small pockets along the sleeves. Similarly, sportsmen suffer the same inconvenience, since they often removes articles of clothing when forcing themselves, rather than wearing alternative clothing. This problem is particularly felt in sporting environments where the items of specialised clothing already have only a few or very small pockets which are of little use.

The aforementioned circumstances lead to a number of drawbacks which, apart from the fact that they may irritate the person who is looking for a small object, may also lead to the said small object being lost because it was not correctly positioned, or even renouncing to carrying the small object about if possible, and delegating another person to carry the object.

The applicant is not aware of alternative proposals to the conditions mentioned above, nor is he aware of accessories dedicated to the same use as the invention contained in this document.

On the other hand, arm-bands are widely used. These accessories, which are used both in sporting environments, such as inflatable arm bands used in swimming, and in building sites for signaling purposes, have the fact that they are ring-shaped in common, so as to fit it around the muscles in the upper arm, in the area close to the shoulder. The ring may be continuous, and in this case is made of elastic material in order to insert an arm through it and be held in position, or it may be of the type which can be opened or closed by means of dedicated fasteners positioned at the opposite ends of the arm-band.

Usually, the latter type of arm-band is made up of a flat, rectangular strap, with at least one side, usually the one that is on view, used to supply a series of indications, which may be a series of letters such as S.O.S. or assistance messages in general, or a figurative message, such as a series of alternating red and white diagonal strips. Another type of strap may have reflective or fluorescent material on the side on view, in order to render the person that wears the said accessory more visible.

The drawback of the arm-bands currently in use is due to the fact that their use is limited to general indication purposes, to warn the people that are getting close to them. A second aspect regards the fact that the strap-type arm-band is not very versatile, in spite of their adherent surface. In fact, the inner surface of the strap-type arm-band does not help it to stick to the surface of the item of clothing over which it is placed whatever the material it is made out of, or to the skin of the arm, which often results in it gradually sliding down along the arm, even if it is fitted on tightly, and it becomes simply irritating to the person that uses it, up to the point that it is eventually taken off.

Therefore, this leads to the user looking for other and better alternatives to the arm-bands described above.

### BRIEF DESCRIPTION OF THE INVENTION

These and other objectives are reached through this invention according to the characteristics in the attached claims, overcoming the problems outlined by means of a high-visibility technical arm-band with at least one pocket, made up of a basically rectangular strap with reciprocal holding means at each end to form a ring shape, and in which the said strap has a surface made out of high-visibility material on the outer side, and to which a pocket, with an opening which may be opened and closed if required, is joined; the said strap, in correspondence with the rear side which adheres to the underlying surface, has a combination of materials which, distributed in a longitudinal manner, are used to improve the friction with the said surface.

In this way, through the creative contribution that has the effect of constituting an immediate technical progress, various advantages are achieved.

Firstly, the strap-type arm-band thus obtained, with the aid of at least one special pocket, allows small objects to be arranged in a rational manner and be immediately at hand, and which are difficult to mislay.

Secondly, the particular format of the materials, foreseen along the inner face of the armband, apart from guaranteeing a high stability of the arm-band itself, means that the arm-band may be used for a large number of applications, independently of the type of underlying surface. The armband may thus be used both over any type of clothing with sleeves or in direct contact with the skin. The use of the said materials with a high friction capacity help to achieve a uniform, perimetral gripping capacity of the arm-band, thus avoiding it slipping down along the arm.

Finally, the side on the outer surface of the arm-band is a high visibility type, thus helping in increasing the safety factor for the person who wears the accessory.

### BRIEF DESCRIPTION OF THE SEVERAL VIEWS OF THE DRAWINGS

These and other advantages will be shown in the following detailed description and attached drawings of at least one preferential solution, the particulars of which are to be considered simply illustrative and not limiting.

FIG. 1 is a view of a strap-type arm-band which is open and in a flat position, and features only the front part.

FIG. 2 is a view of the rear side of the same strap-type arm-band as shown in FIG. 1.

FIG. 3 is a transversal sectional view of the arm-band as shown in FIG. 1, along the a-a axis and with the pocket illustrated in a closed position.

FIG. 4 is a further transversal view of the arm-band as shown in FIG. 1, but with the pocket in a partially open position.

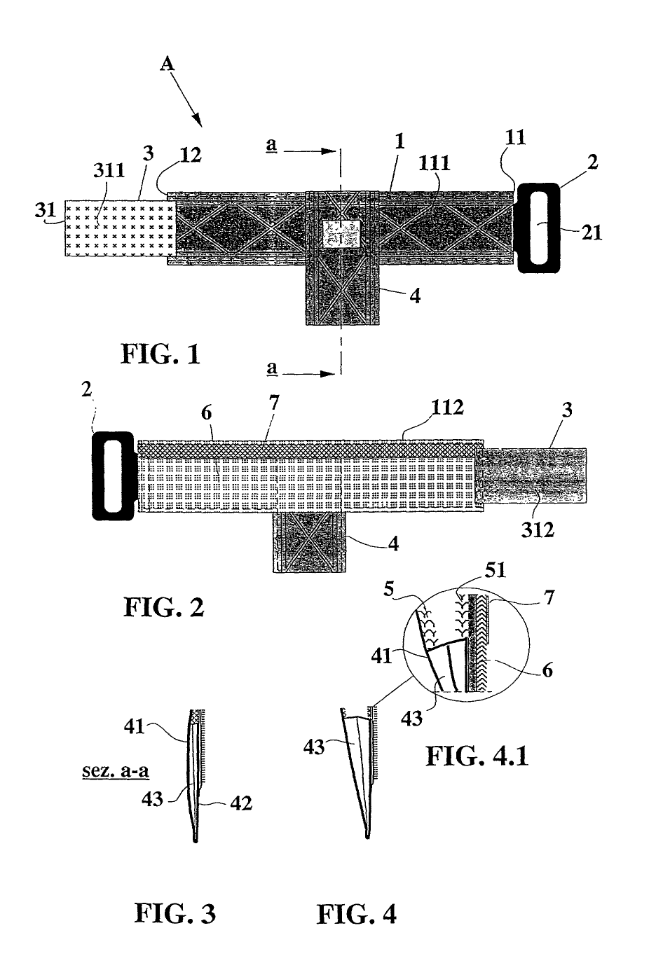

Finally, FIG. 4-1 is a detailed view of the upper end of the pocket as shown in the previous Figures.

### DETAILED DESCRIPTION OF THE INVENTION

The accessory contained herein refers to a strap-type arm-band (A) used to contain small objects, and manufactured in order to make it highly visible, even at night.

Going into detail, it is a strap (1) with a basically rectangular shape, with reciprocal fastening means at its ends (11, 12). The said fastening means include, respectively, a buckle (2) attached firmly to one end (11) of the strap (1), while at the opposite end (12) there is a tongue of material (3) which is firmly attached, and with a Velcro strip on one side (311) of it. In this way, in order to close the ring-shaped strap around the arm, the end (31) of the tongue (3) is passed through the opening (21) in the buckle (2), the arm-band is then tightened according to individual requirements, and the said end (31) that remains is folded over on the side (311) of the tongue (3) that remains before the 11 buckle (2).

The strap (1) has an outer side (111) which is the side on view, and one side on the inside (112). The outer side (111) which is on view is of a high-visibility type, that is to say, it has a support material with a reflective and/or fluorescent surface, and has a quilted effect. The tongue part (3) also has one of its sides (312) which is highly visible, but in this case it is on the opposite side with respect to the side (311) of the strap (1). Consequently, the surface of the tongue (3) with the Velcro (311) faces the same way as the surface (111) of the remaining strap (1).

In correspondence with the outer surface (111), a pocket (4) is attached in the middle. The said pocket (4) is obtained by using the same material as is used for the high-visibility side (111) of the strap (11). Going further into detail, a portion of the strap is folded into a “V” shape, with a front part (41) and a rear part (42). The front part (41) has a small transversal Velcro strip (5) on its peak which corresponds with a second Velcro strip (51) which is locally attached along the upper edge of the side (111) of the strap (1), thus forming a closure. The front part (41) which is folded over the strap (1) is closed on the sides by a bellow-type (43) formation, one on each side, with each one being stitched on the opposite edge with respect to the front part (41) and on the side (111) of the strap. Finally, the rear part (42), continuing on from the front part (41), is fastened to the strap (1).

Regarding the inner side (112) of the strap (11), it is given by a number of materials joined on one side to the high-visibility material which characterises the outer side (111). Going into detail, the entire surface is covered by material such as soft, absorbent cloth (6), such as terry-cloth, with a plastic or rubber material (7) with high gripping capacities along the upper edge.

1. Technical arm-band, characterised by the fact that it includes a strap (1) with reciprocal fastening means (2, 3) at each one of its ends (111, 12) in order to form a ring, and in which the said strap (11) has at least one pocket (4) in correspondence with the outer side (111) which is the side on view.

2. Technical arm-band, according to claim 1, characterised by the fact that at least the side on view (111) of the strap (1) is made from high visibility material,

3. Technical arm-band, according to claims 1 and 2, characterised by the fact that the inner side (12) of the strap (1) is made from at least one of material (7) with high gripping capacities.

4. Technical arm-band, according to the previous claims, characterised by the fact that the entire surface of the inner side (112) is covered by a material such as soft, absorbent cloth (6), such as terrycloth, with a strip of plastic or rubber material (7) with high gripping capacities along the upper edge.

5. Technical arm-band, according to the previous claims, characterised by the fact that the pocket (4) is made from the same material used to make the high-visibility (111) surface of the strap (1), and that it has a portion of the strap folded into a “V” (shape, with a front part (41) and a rear part (42); the said front part (41) is folded over the strap (1) and is closed on the sides and joined by two bellow-type (43) formations, one on each side, with each one being attached on one side to the said front part (41) and on the other side to the side (111) of the strap.

6. Technical arm-band, according to the previous claims, characterised by the fact that the pocket (4) may be opened and closed, and that it has a small transversal Velcro strip (5) on the peak of the front part (41) which corresponds with a second Velcro strip (51) which is locally attached along the upper edge of the side (111) of the strap (1).
